(19) United States
(12) Patent Application Publication
(10) Pub. No.: US 2018/0194360 A1
(43) Pub. Date: Jul. 12, 2018

(54) PROTECTION FROM HEAT IN VEHICLES

(71) Applicants: William Robinson HILL, Prescott, AZ (US); Michele Frances WOLFGRAM, Prescott, AZ (US); Elizabeth Ann WAITES, Albuquerque, NM (US)

(72) Inventors: William Robinson HILL, Prescott, AZ (US); Michele Frances WOLFGRAM, Prescott, AZ (US); Elizabeth Ann WAITES, Albuquerque, NM (US)

(21) Appl. No.: 15/401,058

(22) Filed: Jan. 8, 2017

(51) Int. Cl. B60W 50/00; B60Q 5/00; E05F 15/71; E05F 15/72; G01N 33/00

(52) U.S. Cl. CPC B60W 50/0098; B60Q 5/005; E05F 15/71; H04W 4/22; G01N 33/004; E05Y 2900/55; B60W 2400/00; E05F 15/72

(57) ABSTRACT

A technique of attempting to protect at least one living entity from heat build-up in a vehicle. The technique includes detecting an increase or a level of heat in the vehicle, detecting a presence of the living entity in the vehicle, and taking an action responsive to the steps of detecting the increase or the level of heat in the vehicle and detecting the presence of the living entity in the vehicle. The detecting functions may occur while the vehicle is otherwise turned off. Detecting the presence of the living entity may include detecting a carbon dioxide level or a change in the carbon dioxide level in the vehicle. Taking the action may include activating a horn of the vehicle, lowering at least one window of the vehicle, and/or sending a message such as a text message to the owner/operator of the vehicle and/or emergency services. Also, associated systems.

### CROSS-REFERENCE TO RELATED APPLICATION

Not applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISK APPENDIX

Not Applicable

### BACKGROUND

The present disclosure generally relates to attempting to protect children, pets, and/or other living entities from heat build-up in a vehicle.

The article “Proposed state bill: Passersby could break into hot cars to save kids, pets” © Copyright 2016 The Daily Courier and Western News & Info®, Inc. with all rights reserved (the “Proposed Bill Article”) describes hazards to children, pets, and/or other living entities from heat build-up in a vehicle: Numerous other articles and documents have described this potentially and actually deadly hazard. For example, see also the article from Golden Gate Weather Services titled “Heatstroke Deaths of Children in Vehicles” Copyright © 2016 (the “Heatstroke Article”). These articles are cited in an Information Disclosure Statement associated with this filing and are hereby incorporated by reference as if fully set forth herein.

### SUMMARY

Aspects of the subject technology include techniques of attempting to protect at least one living entity from heat build-up in a vehicle. In some aspects, the subject technology may be embodied in a method that includes detecting an increase or a level of heat in the vehicle, detecting a presence of the living entity in the vehicle, and taking an action responsive to detecting the increase or the level of heat in the vehicle and detecting the presence of the living entity in the vehicle.

Detecting the increase or the level of heat in the vehicle and detecting the presence of the living entity in the vehicle preferably may occur while the vehicle is otherwise turned off. Detecting the presence of the living entity in the vehicle may include detecting a carbon dioxide level or a change in the carbon dioxide level in the vehicle.

In various aspects, taking the action may include activating a horn of the vehicle, lowering at least one window of the vehicle, and/or sending a message. The message may be a text message and may be sent to an owner or operator of the vehicle and/or an emergency service provider. In the case that the message is sent to an emergency service provider, the message may include location information regarding the vehicle, for example as determined by a GPS system.

In some aspects, multiple actions may be taken. For example, if detected conditions remain after a period of time from taking one action (e.g., messaging the owner or operator), another action may be taken (e.g., messaging emergency services).

The subject technology also includes systems and/or devices that implement the foregoing techniques.

This brief summary has been provided so that the nature of the invention may be understood quickly. Additional steps and/or different steps than those set forth in this summary may be used. A more complete understanding of the invention may be obtained by reference to the following description in connection with the attached drawings.

### BRIEF DESCRIPTION OF THE DRAWINGS

FIG. 1 is a block diagram illustrating systems and/or devices according to aspects of the subject technology.

FIG. 2 illustrates techniques according to aspects of the subject technology.

FIG. 3 illustrates a specific example of a technique according to aspects of the subject technology.

FIGS. 4 and 5 show experimental results related to aspects of the subject technology.

### DETAILED DESCRIPTION

As mentioned in the Proposed Bill Article, Arizona SB 1001 requires a “good faith belief” a child or pet is “in imminent danger of suffering physical injury or death” unless removed from the vehicle for a bystander to act. However, just a good faith belief would not be enough under SB 1001. Before breaking into a vehicle, the bystander would have to notify authorities such as the police, emergency medical services, or pet control.

The Proposed Bill Article and the Heatstroke Article also noted many living entities including children and pets die every year when left in a parked vehicle, especially during hot days. One noted issue that leads to this hazard is bystanders often do not notice that a living entity is in the parked vehicle. That said, bystanders are often the first people to notice such.

Another noted issue is bystanders may be reluctant to intervene even if they do notice such, for example in fear of legal consequences. While some legislators have and/or have attempted to pass laws to protect bystanders who do take action, some law enforcement officials are “uneasy” with those attempts. One example is Prescott Valley Police Chief Bryan Jarrell mentioned in the Proposed Bill Article, who was quoted as saying “Police officers have a great deal of training and I would be concerned that citizens may overreact, or react too quickly, resulting in damage to a person's vehicle” and “Another concern is that although a person may be exempt from liability, that is not going to help them when an angry car owner confronts someone who just broke out their window.”

The subject technology attempts to address at least some of these and/or other issues in an attempt to save lives.

Briefly, techniques according to aspects of the subject technology include attempting to protect at least one living entity from heat build-up in a vehicle. In some aspects, the subject technology may be embodied in a method that includes detecting an increase or a level of heat in the vehicle, detecting a presence of the living entity in the vehicle, and taking an action responsive to detecting the increase or the level of heat in the vehicle and detecting the presence of the living entity in the vehicle.

Detecting the increase or the level of heat in the vehicle and detecting the presence of the living entity in the vehicle preferably may occur while the vehicle is otherwise turned off. Detecting the presence of the living entity in the vehicle may include detecting a carbon dioxide level or a change (e.g., increase) in the carbon dioxide level in the vehicle.

In various aspects, taking the action may include activating a horn of the vehicle, lowering at least one window of the vehicle, and/or sending a message. The message may be a text message and may be sent to an owner or operator of the vehicle and/or an emergency service provider. In the case that the message is sent to an emergency service provider, the message may include location information regarding the vehicle, for example as determined by a GPS system.

In some aspects, multiple actions may be taken. For example, if detected conditions remain after a period of time from taking one action (e.g., messaging the owner or operator), another action may be taken (e.g., messaging emergency services).

The subject technology also includes systems and/or devices that implement the foregoing techniques.

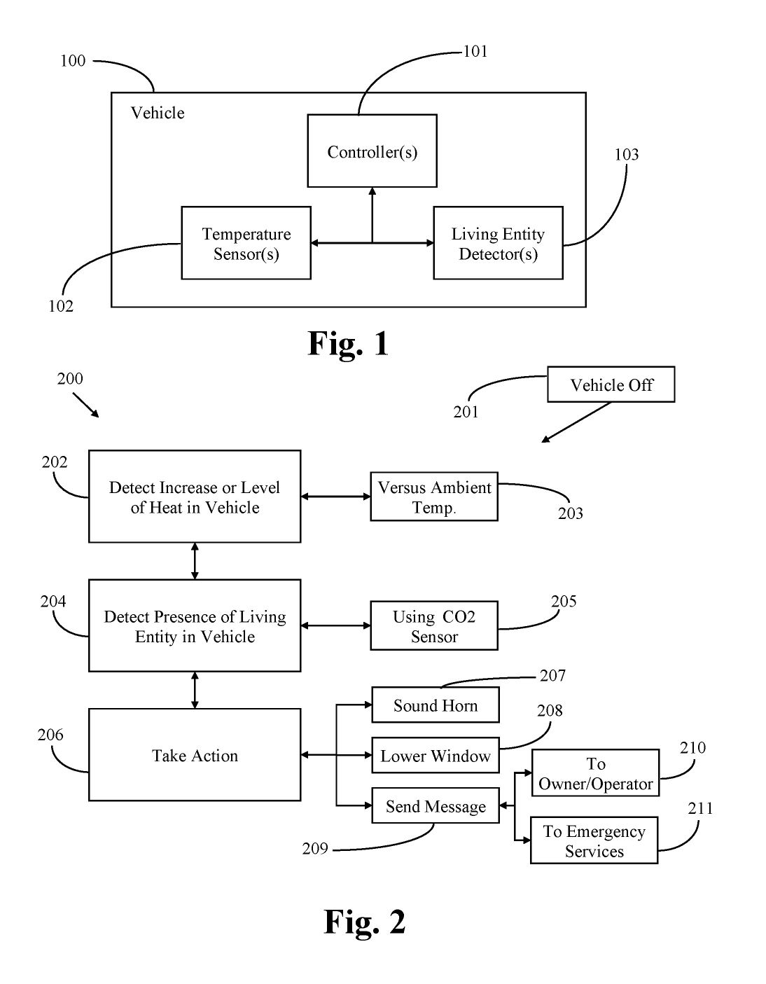

In more detail, FIG. 1 is a block diagram illustrating systems and/or devices according to aspects of the subject technology. Vehicle 100 may be any type of vehicle, for example a car, pickup truck, SUV, semi-truck, boat, airplane, or the like. Modern versions of such vehicles include controller(s) 101 that may control various functions of the vehicle. These controller(s) typically are computing devices as disclosed below.

FIG. 1 also shows temperature sensor(s) 102 and life entity detector(s) 103. These elements preferably communicate with controller(s) 101.

Temperature sensor(s) 102 may be any form of temperature sensor. Examples include but are not limited to thermometers that generate a signal regarding temperatures inside and outside of the vehicle.

Life entity detector(s) 103 may be any form of life entity detector(s). In some embodiments, life entity detector(s) 103 measure a level or change (e.g., increase) of a level of carbon dioxide (CO2) within the vehicle. The detector(s) may be non-dispersive infrared (NDIR) detector(s), which can be purchased at present in quantity for less than $150. For example, the SensAir S-8 CO2 monitor may be purchased in quantity for less than $50 and includes associated logic circuits, autozero functions, and power supplies that may facilitate connection to higher-order logic systems. One preferred feature of this NDIR system is self error checking.

The subject technology is not limited to NDIR detector(s). For example, other possible life entity detector(s) include but are not limited to infrared motion detectors, O2 depletion and/or level detectors, weight sensors, and/or sound detectors. A combination of these and/or other life detectors may be used.

Some and/or all element(s) shown in FIG. 1 may already exist in current vehicles. However, the elements are not believed to communicate with each other according to aspects of the subject technology in order to protect living entities (e.g., children, elder individuals, pets, etc.) as set forth herein. This paragraph is not an admission that some and/or all elements shown in FIG. 1 or elsewhere in this document do exist in current vehicles.

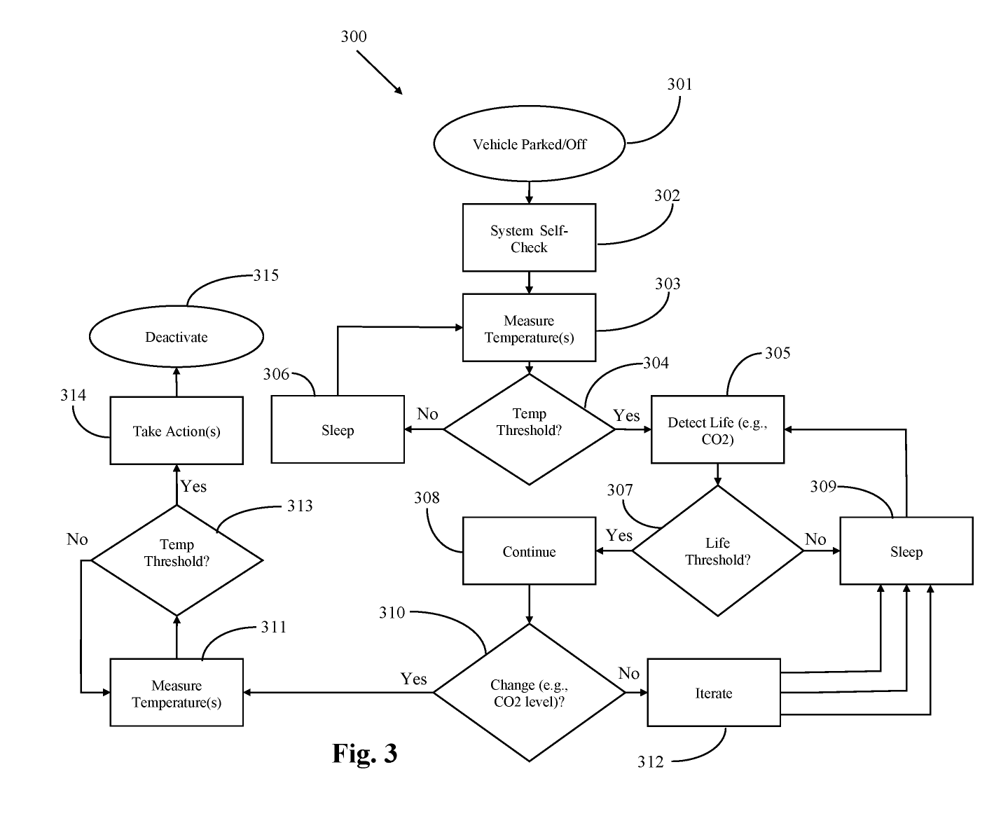

FIG. 2 illustrates techniques 200 according to aspects of the subject technology. A vehicle is turned off in step 201. Alternative, the step may represent an air conditioner, ventilation, or other system being turned off.

A level of heat and/or temperature in the vehicle is detected in step 202, for example using temperature sensor(s) 102. A comparison with ambient (e.g., outside) temperature may be performed in step 203.

A presence of a living entity such as a child, elderly person, pet, or the like may be detected in step 204. This detection preferably occurs via a CO2 sensor, as illustrated in step 205, or via any other life detecting sensor as described above.

In step 206, an action may be taken responsive to steps 202, 203, 204, and/or associated steps. For example, if a temperature inside the vehicle exceeds a pre-defined threshold such as 75 degrees Fahrenheit or a difference between an ambient temperature exceeds 10 degrees Fahrenheit, and life or potential life is detected in the vehicle, action may be take in step 206.

Examples of possible actions include but are not limited to sounding a horn in step 207, lowing one or more windows in step 208, and/or sending a message in step 209. The message may be a text, (possibly automated) voice message, email, or any other form of message. The message may be sent to an owner and/or operator of the vehicle in step 210 and/or to emergency services in step 211. The message sent in step 211 may be via 911 or some other number and/or address to local police, fire department(s), pet control, hospital(s), one or more vehicle service companies such as AAA, and/or the like.

Of note, sounding a horn under step 207 may afford bystander(s) with a good faith belief that a child, elderly person, pet, or other living entity is “in imminent danger of suffering physical injury or death” unless removed from the vehicle. Sending the message under step 211 may also relieve such bystander(s) of a duty to notify authorities of a potential danger to a living entity before acting.

In some aspects, step 206 and/or the associated steps may be repeated and/or initiated after a certain period of time. Different of the associated steps may be initiated and/or repeated. For example, if the owner and/or operator does not respond a period of time (e.g., 5 or 10 minutes) after step 210, a message may be sent to emergency services in step 211.

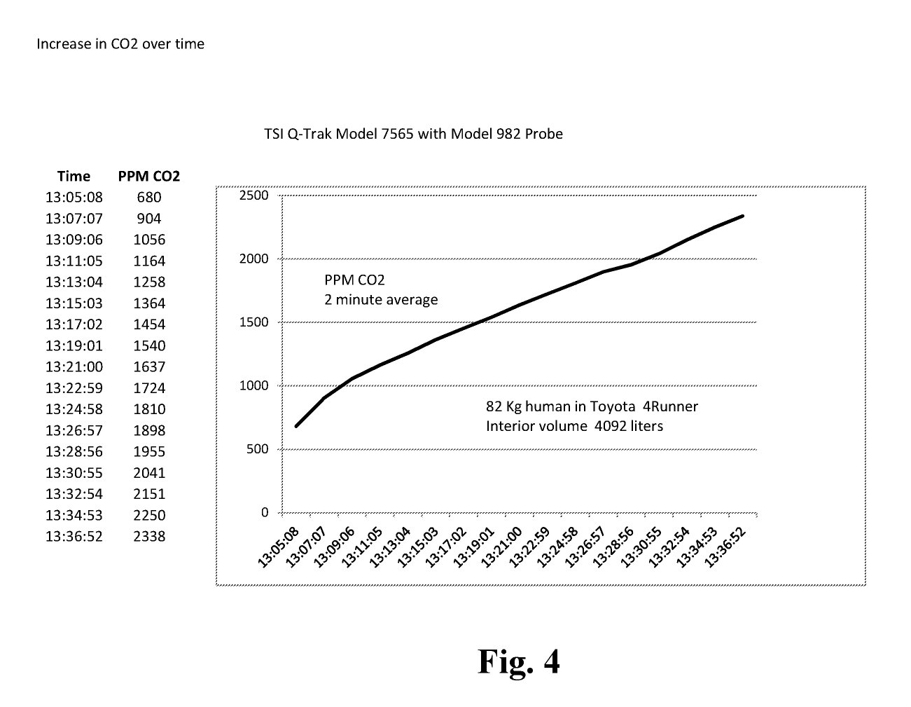

FIG. 3 illustrates example 300 of a technique according to aspects of the subject technology. The technology is not limited to the details shown in and described with respect to FIG. 3.

In step 301, a vehicle is parked and/or turned off. Alternatively, the subject technology may be used even while the vehicle is not parked, turned off, and/or otherwise.

A system self-check may be performed in step 302.

Temperature(s), for example ambient (external) and/or internal vehicle temperatures, may be measured in step 304. If the ambient and/or internal vehicle temperature(s) exceed a threshold, flow may proceed from step 304 to step 305. Likewise, if a difference between the ambient and internal temperatures exceeds a threshold, flow may proceed to step 305. Additionally, if an increase in internal temperature (e.g., from 75 to 85 degrees Fahrenheit) over a period of time exceeds a threshold, flow may also proceed to step 305. These thresholds preferably are pre-defined, but may be user defined or otherwise defined. These and other thresholds and/or parameters may be used to trigger flow proceeding to step 305, either alone or in combination.

If flow does not proceed to step 305, flow may proceed to a sleep state in step 306. The sleep state may last for a period of time, for example 4, 5, or 10 minutes and/or any other period of time. The period of time preferably is predetermined, but may be user defined or otherwise defined. The sleep state may help conserve battery power. When the sleep state expires, flow preferably returns to step 303.

If flow proceeds to step 305, initiation of detecting and/or detecting life within the vehicle may occur in that step. Flow may the proceed to step 307, where various thresholds may be applied. For example, detection of an increase in CO2 within the vehicle may indicate a presence of life. For another example, detection of some level such as 450 ppm of CO2 within the vehicle may indicate a presence of life. A lower level of detected CO2 may indicate the presence of potential life. A precipitous drop in a CO2 level after reaching some threshold may indicate an emergency regarding possible expiration of life within the vehicle. As discussed above, other life detection techniques and/or devices may be used.

If life and/or potential life is detected in the vehicle, flow preferably proceeds from step 307 to step 308. Otherwise, flow may proceed to a sleep state in step 309. The sleep state may last for a period of time, for example 4, 5, or 10 minutes and/or any other period of time. The period of time preferably is predetermined, but may be user defined or otherwise defined. The sleep state may help conserve battery power. When the sleep state expires, flow preferably returns to step 305. Alternatively, flow may return to step 303 or some other step.

Returning to step 308, continued monitoring for life and/or potential life may be performed in this step. Any form of life detection may be used in step 308. Flow may then proceed to step 310 for determination of how to proceed.

For example, a change in the CO2 level in the vehicle may have been detected in step 305, 307, 308, 310, or any other step. An increase of CO2 in the vehicle may indicate and/or confirm a presence of a living entity in the vehicle. Flow may then proceed from step 310 to step 311. Otherwise, flow may proceed to step 312 for iteration of various other steps.

For another example, a decrease in CO2 level from some pre-defined threshold may have been detected in step 308, possibly indicating an emergency regarding potential expiration of life in the vehicle. Emergency action may then be initiated, for example sending a message as described above with respect to steps 209 to 211.

Ambient and/or internal temperatures may be further measured in step 311. If the temperature(s) and/or comparison of temperatures exceed a threshold in step 313, flow may then proceed to step in step 314. In that step, various actions may be taken in an effort to protect a living entity in the vehicle, for example as described above with respect to steps 206 to 211. Those and/or other steps may be taken as well.

The techniques shown in FIG. 3 may end in step 315, for example if the vehicle is started, a function is activated in the vehicle to stop the techniques (e.g., the owner/operator, a bystander, or an emergency service provider pushes an “off” button), etc.

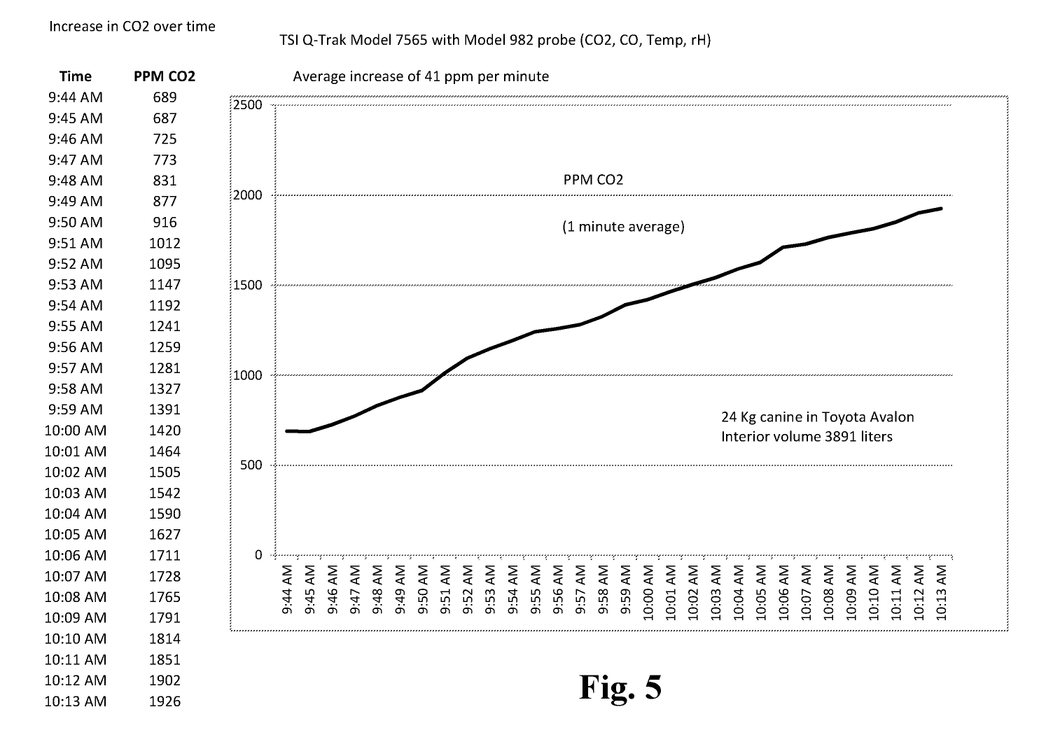

FIG. 4 shows a graph of increased CO2 levels resulting from presence of a human within a vehicle. FIG. 5 shows a graph of increased CO2 levels resulting from the presence of an animal. FIGS. 4 and 5 reflect results of controlled experiments observed during cool and/or cold weather with no possibility of harm to the human or the animal. The figures verify that a presence of a living entity in a vehicle may be detected by a change (e.g., increase) in CO2 levels. Again, the subject technology is not limited to using measurement of CO2 levels to detect presence of one or more living entities within a vehicle.

Aspects of the subject technology may be performed by one or more computing devices such as controller 101 in FIG. 1. The computing device(s) preferably include at least a tangible computing element. Examples of a tangible computing element include but are not limited to a microprocessor, application specific integrated circuit, programmable gate array, memristor based device, and the like. A tangible computing element may operate in one or more of a digital, analog, electric, photonic, and/or some other manner.

Examples of a computing device include but are not limited to a vehicle's controller. The vehicle may also communicate with a mobile computing device such as a smart phone or tablet computer, a wearable computing device (e.g., Google® Glass), a laptop computer, a desktop computer, a server, a client that communicates with a server, a smart television, a game console, a part of a cloud computing system, a virtualized computing device that ultimately runs on tangible computing elements, or any other form of computing device. One or more of such devices may perform one or more of the functions described herein. Communication between computing devices may be through one or more other computing devices and/or networks. Additionally, some operations may be considered to be performed by multiple such computing devices.

The invention is in no way limited to the specifics of any particular embodiments and examples disclosed herein. For example, the terms “aspect,” “example,” “preferably,” “alternatively,” “e.g.,” “etc.,” and the like denote features that may be preferable but not essential to include in some embodiments of the invention. In addition, details illustrated or disclosed with respect to any one aspect of the invention may be used with other aspects of the invention. Additional elements and/or steps may be added to various aspects of the invention and/or some disclosed elements and/or steps may be subtracted from various aspects of the invention without departing from the scope of the invention. Singular elements/steps imply plural elements/steps and vice versa. Some steps may be performed serially, in parallel, in a pipelined manner, or in different orders than disclosed herein. Many other variations are possible which remain within the content, scope, and spirit of the invention, and these variations would become clear to those skilled in the art after perusal of this application.

1. A method of attempting to protect at least one living entity from heat build-up in a vehicle, comprising:
detecting an increase or a level of heat in the vehicle;
detecting a presence of the at least one living entity in the vehicle; and
taking an action responsive to detecting the increase or the level of heat in the vehicle and detecting the presence of the at least one living entity in the vehicle;
wherein detecting the presence of the at least one living entity in the vehicle comprises detecting a change in a carbon dioxide level in the vehicle.

2. The method as in claim 1, wherein detecting the increase or the level of heat in the vehicle and detecting the presence of the at least one living entity in the vehicle occur while the vehicle is otherwise turned off.

3. The method as in claim 1, wherein detecting the increase or the level of heat in the vehicle is performed with respect to ambient temperature.

4. The method as in claim 1, wherein taking the action comprises activating a horn of the vehicle.

5. The method as in claim 1, wherein taking the action comprises lowering at least one window of the vehicle.

6. The method as in claim 1, wherein taking the action comprises sending a message.

7. The method as in claim 6, wherein the message comprises a text message.

8. The method as in claim 6, wherein the message is sent to an owner or operator of the vehicle.

9. The method as in claim 6, wherein the message is sent to an emergency service provider and includes location information regarding the vehicle.

10. The method as in claim 1, further comprising taking another action or repeating taking the action a period of time after taking the action.

11. A system that attempts to protect at least one living entity from heat build-up in a vehicle, comprising:
a device that detects an increase or a level of heat in the vehicle;
a device that detects a presence of the at least one living entity in the vehicle; and
a computing device that takes an action responsive to detection of the increase or the level of heat in the vehicle and detection of the presence of the at least one living entity in the vehicle;
wherein the device that detects a presence of the at least one living entity in the vehicle comprises a carbon dioxide sensor, and the detection of the presence of the at least one living entity in the vehicle comprises detecting a change in a carbon dioxide level in the vehicle.

12. The system as in claim 11, detecting the increase or the level of heat in the vehicle and detecting the presence of the at least one living entity in the vehicle occur while the vehicle is otherwise turned off.

13. The system as in claim 11, wherein detecting the increase or the level of heat in the vehicle is performed with respect to ambient temperature.

14. The system as in claim 11, wherein taking the action comprises activating a horn of the vehicle.

15. The system as in claim 11, wherein taking the action comprises lowering at least one window of the vehicle.

16. The system as in claim 11, wherein taking the action comprises sending a message.

17. The system as in claim 16, wherein the message comprises a text message.

18. The system as in claim 16, wherein the message is sent to an owner or operator of the vehicle.

19. The system as in claim 16, wherein the message is sent to an emergency service provider and includes location information regarding the vehicle.

20. The system as in claim 11, wherein the system further takes another action or repeats taking the action a period of time after taking the action.

21. The method as in claim 1, wherein detecting the change in the carbon dioxide level in the vehicle further comprises detecting an increase in the carbon dioxide level.

22. The method as in claim 1, wherein detecting the change in the carbon dioxide level in the vehicle further comprises detecting a drop in the carbon dioxide level, possibly indicating expiration of the at least one living entity in the vehicle.

23. The system as in claim 11, wherein detecting the change in the carbon dioxide level in the vehicle further comprises detecting an increase in the carbon dioxide level.

24. The system as in claim 11, wherein detecting the change in the carbon dioxide level in the vehicle further comprises detecting a drop in the carbon dioxide level, possibly indicating expiration of the at least one living entity in the vehicle.
